(19) United States
(12) Patent Application Publication
(10) Pub. No.: US 2026/0325341 A1
(43) Pub. Date: Oct. 22, 2026

(54) SMART PILLOW DEVICE WITH ANTISNORING CAPABILITY AND METHOD FOR PERFORMING ANTISNORING FOR USER

(71) Applicant: BO PI, SAN DIEGO, CA (US)

(72) Inventors: BO PI, SAN DIEGO, CA (US); IRENE PI, SAN DIEGO, CA (US)

(21) Appl. No.: 19/182,321

(22) Filed: Apr. 17, 2025

(51) Int. Cl. A61F 5/56; A47G 9/10

(52) U.S. Cl. CPC A61F 5/56; A47G 9/1027

(57) ABSTRACT

A smart pillow device with antisnoring capability is provided in the present application The smart pillow device includes a plurality of airbags and an airbag control module; the airbag control module includes a case and a plurality of airflow control units received in the case and connected to the plurality of airbags respectively. Each of the airflow control unis comprises a piezoelectric pump. an air connector and an air pressure sensor, the piezoelectric pump is connected to a corresponding one of the airbags via the air connector, and the air pressure sensor is connected to the air connector for detecting an air pressure of the airbag; the piezoelectric pump is driven to inflate or deflate the airbag independently according to an air pressure of the airbag. A method for performing antisnoring for a user by using a smart pillow device is also provided in the present application.

### TECHNICAL FIELD

This present application generally relates to sleep health assistance field, and more particularly, to a smart pillow device with an antisnoring capability and a method for performing antisnoring for a user by using the smart pillow device.

### BACKGROUND

Snoring is a common sleep phenomenon for most people, especially for the people with obesity, fatigue, or drinking. Snoring is generally caused by vibration of soft tissues around a respiratory tract of a person during sleep; this vibration may be amplified by a resonance effect of the throat and mouth, and finally produce a common-known snoring sound.

Generally speaking, snoring occurs when a respiratory tract becomes narrow or compressed which causes an airflow in the respiratory tract to be blocked. There are two common types of snoring, pathological snoring and physiologic snoring. Pathological snoring is mainly caused by physical diseases; for example, rhinitis and sinusitis can lead to nasal obstruction and further induce respiratory tract narrowing, which needs timely medical treatment. However, most of the snoring is physiologic snoring, for example, obesity may cause a pharyngeal adipose layer to be thick and narrows the respiratory tract, drinking or long-term fatigue may also cause pharyngeal muscles to be relax during sleep, which further lead to physiologic snoring; in addition, sleep posture can also affect the respiratory tract, especially when a person lies on the back, his or her tongue and soft palate may fall and narrows the respiratory tract, which further increases a possibility of snoring.

Snoring is usually accompanied by sleep breathing disorder or even result in hypoxemia and sleep apnea syndrome; long-term snoring will lead to poor sleep quality and affect people's mental state and health. Therefore, if a person suffers snoring, it is necessary to intervene as early as possible.

As known by many people, these physiologic snoring can be alleviated by adjusting a sleep posture. An available solution for alleviating physiologic snoring is to use a smart antisnoring pillow during sleep, the smart pillow is inflatable to change heights of different airbags therein and assist a user in adjusting his or her sleep posture, such as turning the user's head to different orientations. In this manner, a width of the respiratory tract of the person can be adjusted and thus alleviating the physiologic snoring. However, the conventional smart pillow generally has a complicated internal structure and airflow control mechanism and thus needs to take an unduly large space, which is not convenience for use.

It is, therefore, desired to provide a new smart pillow device with antisnoring capability, which can be used to for alleviating snoring of a user during sleep.

### SUMMARY

The examples of implementations described in the present application provide a smart pillow device with a plurality of airbags each of which is controlled by an independent airflow control unit respectively, the smart pillow device is capable of assisting a user in antisnoring during sleep for improving the user's sleep quality and further improving the mental state and health of the user.

In an implementation according to one aspect of the present application, a smart pillow device with antisnoring capability includes a plurality of airbags and an airbag control module, the airbag control module includes a case and a plurality of airflow control units received in the case and connected to the plurality of airbags respectively. Each of the airflow control unis includes a piezoelectric pump, an air connector and an air pressure sensor, the piezoelectric pump is connected to a corresponding one of the airbags via the air connector, and the air pressure sensor is connected to the air connector for detecting an air pressure of the airbag. The piezoelectric pump is driven to inflate or deflate the airbag independently according to an air pressure of the airbag.

In an implementation according to another aspect of the present application, a method for performing antisnoring for a user by using a smart pillow device is provided, the smart pillow device includes a plurality of airbags and a plurality of airflow control units, each of the airflow control units includes a piezoelectric pump and an air pressure sensor connected to a corresponding airbag, the method includes: estimating at least one head-turn parameter for the user during sleep; determining target heights of the plurality of airbags based on the at least one head-turn parameter; calculating target air pressure values in accordance with the target heights of the airbags; and driving each of the piezoelectric pumps independently to inflate or deflate a corresponding one of the airbags according to the target air pressure value of the airbag.

The above and other aspects and features are described in greater detail in the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic diagram of a typical smart pillow device, which includes a main control box with a total air pump.

FIG. 2 is an exploded diagram of the main control box of the smart pillow device of FIG. 1.

FIG. 3 is a structural diagram of the airbag control module of the smart pillow device of FIG. 1.

FIG. 4 is an enlarged view of the airbag control module of FIG. 3.

FIG. 5 is a schematic diagram of a smart pillow device with antisnoring capability according to an exemplary embodiment of the present application.

FIG. 6 is an enlarged view of an airbag control module of the smart pillow device of FIG. 5, the airbag control module including a plurality of airflow control units.

FIG. 7 is a schematic view of an exemplary airflow control unit of the airbag control module of FIG. 6.

FIG. 8 is a circuit diagram of the airbag control module of FIG. 6, illustrating a circuit connection between the airbags and the airflow control units.

FIG. 9 illustrates an arrangement of piezoelectric pumps of the air flow control units of FIG. 6.

FIG. 10 is a flow chat illustrating a method for performing antisnoring for a user by using the smart pillow device of FIG. 5.

### DETAILED DESCRIPTION

Detail technical solutions in various embodiments of the present application will be described hereinafter in conjunction with the attached drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following description is directed to a number of various embodiments. The described embodiments, however, may be implemented and/or varied in many different ways. For example, the described embodiments may be implemented as a smart pillow device with a plurality of airbags each of which being controlled with an independent airflow control unit respectively, the airflow control unit can inflate or deflate the corresponding airbag to adjust a height of the airbag, so as to assist a user to implement a head-turn activity and reduce a snoring phenomenon for the user during sleep, and thus can improve the user's sleep quality and further improve the mental state and health of the user.

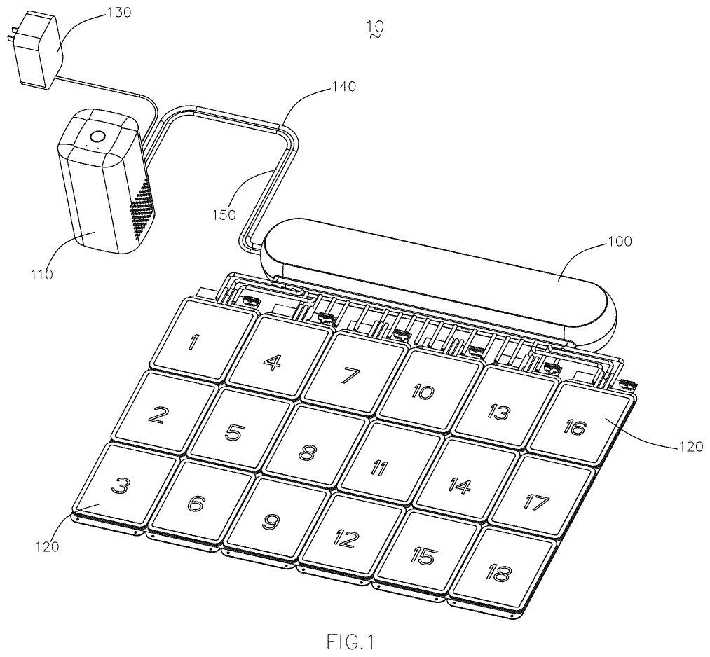

FIG. 1 illustrates a schematic diagram of a typical smart pillow device 10. The smart pillow device 10 includes a main control box 110, an airbag control module 100 connected to the main control box 110, and a plurality of airbags 120 connected to the airbag control module 100 respectively. The main control box 110 may be connected to a power supply via a power adaptor 130 to receive a power voltage from the power supply. The airbag control module 100 and the plurality of airbags 120 can be integrated within a pillow body that is configured for the user's head to be rest on during sleep.

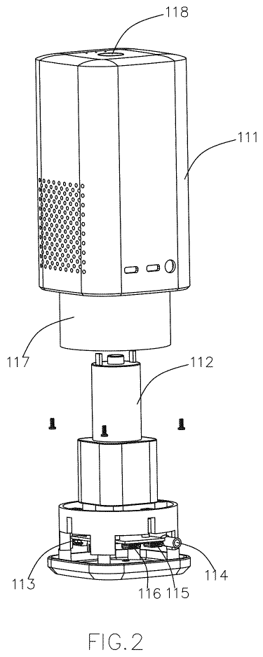

Referring also to FIG. 2, the main control box 110 includes a housing 111, a total air pump 112 and a printed circuit board assembly (PCBA) 113. The total air pump 112 and the PCBA 113 are electrically coupled to each other and are received in the housing 111. The PCBA 113 includes an air pump driver for driving the total air pump 112 to provide a total airflow to the airbag control module 100 through a main air output 114. The main air output 114 is connected to the airbag control module 100 via an air pipe 140, the air pipe 140 is used for providing airflow transmission between the total air pump 112 and the airbag control module 100.

The PCBA 113 may further include a control circuit which is electrically coupled to the airbag control module 100 through a circuit port 115; the circuit port 115 may for example be a USB-C port, which is electrically coupled to the air bag control unit 100 via an USB-C line 150. The USB-C line 150 is configured for providing signal interaction between the PCBA 113 and the airbag control module 100. In addition, a power terminal 116 can also be arranged on the PCBA 113, which is configured for connecting with the power adapter 130 to receive the power voltage.

When the total air pump 112 is driven to generate the airflow, an operation noise may be produced and influence the user's sleep. To reduce the impact of the operation noise, the main control box 110 may further include a soundproofing foam 117 which is also received in the housing 111 and arranged surrounding the total air pump 112, as illustrated in FIG. 2. Furthermore, a switch 118 is further formed on a top surface of the housing 111; the switch 119 is electrically coupled to the PCBA 113 to control an on/off state of the main control box 110.

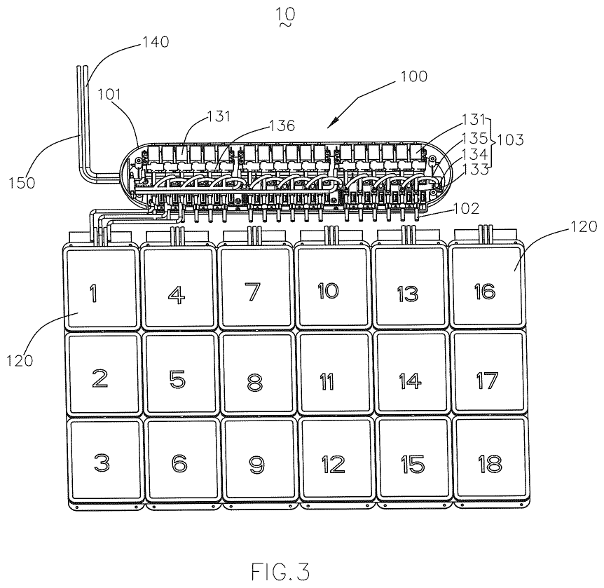

FIG. 3 is a structural diagram of the airbag control module 100 of the smart pillow device 10 of FIG. 1, which also illustrates the connection between the airbag control module 100 and the airbags 120. The airbag control module 100 is configured as a box-like structure with a plurality of air outlets 102; the air outlets 102 are further connected to the plurality of airbags 120 respectively. Specifically, the airbag control module 100 includes a case 101 and a plurality of airflow control units 103 received in the case 101; each of the airflow control units 103 is connected to a respective one of the airbags 120 via a corresponding air outlet 102, and is configured for controlling an inflating or deflating state of the airbag 120 in accordance with, for example, an air pressure of the corresponding airbag 120 as well as a sleep posture of the user of the smart pillow device.

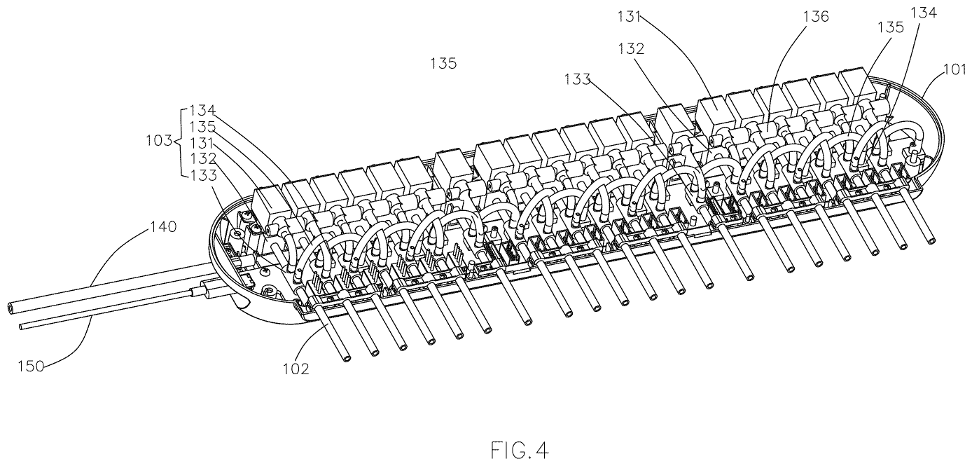

Referring also to FIG. 4, an enlarged view of the airbag control module 100 is shown. Each of the airflow control units 103 includes an electromagnetic intake valve 131, an electromagnetic air release valve 132, a four-way pipe 133 and an air pressure sensor 134. The four-way pipe 133 includes a first end connected to the corresponding air outlet 102, a second end connected to the electromagnetic air release valve 132, a third end connected to the electromagnetic intake valve 131, and a fourth end connected to the air pressure sensor 134. In particular, all the electromagnetic intake valves 131 of the plurality of airflow control units 103 are arranged in a row, and may be divided into several groups, which are supplied with the airflow from the air pipe 140 via a pipe bus 136. Similarly, all the electromagnetic air release valves 132 are located under the electromagnetic intake valves 131 and also arrange in a row. Furthermore, the fourth end of the four-way pipe 133 and the air pressure sensor 134 may be connected preferably through an arc-shaped pipe 135, the arc-shaped pipe 135 may have a sufficient length to avoid from suffering undesired bending.

The air pressure sensor 134 of the airflow control unit 103 may be disposed next to an adjacent four-way pipe 133, and the arc-shaped pipe 135 is arranged to cross over the adjacent four-way pipe 133. In addition, to make the component arrangement in the airbag control module 100 more compact, in every two adjacent four-way pipe 133, one is arranged parallel to the pipe bus 136, and the other one is arranged diagonally in relative to the previous one, as illustrated in FIG. 4.

In operation, each of the air pressure sensors 134 of the airflow control units 103 can detect an air pressure of a corresponding air bag 120; the air pressure detection result of the air pressure sensor 134 may be used for determining whether the corresponding one of the airbags 120 needs to inflated or deflated. Moreover, the main control box 110 drives the total air pump 112 to generate an airflow, and the airflow is outputted to the airbag control module 100 via the air pipe 140. In the airbag control module 100, each of the electromagnetic intake valves 131 and the electromagnetic air release valve 132 in the airflow control units 113 are controlled to be turned on or turned off according to the air pressure of the corresponding airbag 120, so as to change a height of the airbag 120 correspondingly.

For example, when it is determined that the airbag 120 needs to be inflated, the electromagnetic intake valve 131 of the corresponding airflow control unit 103 can be controlled to be turned on, so that the airflow provided from the air pump 112 can be transmitted to the airbag 120 via the electromagnetic intake valve 131 to increase a height of the airbag 120. In contrast, when it is determined that the airbag 120 needs to be deflated, the electromagnetic intake valve 131 can be turned off and the electromagnetic air release valve 132 is controlled to be turned on, so that the air in the airbag 120 can be released from the electromagnetic air release valves 132, and thus reduce the height of the airbag 120. By changing the heights of the airbags 120 in the smart pillow device 10, a sleep posture of the user can be adjusted during sleep, for example, the height changes of different airbags 120 can prompt the user to turn his or her head to an expected orientation. Therefore, the undesired snoring of the user can be alleviated, and the sleep quality of the user can be improved.

In the above-described smart pillow device 10, a single total air pump 112 is used for providing a main airflow to the airbag control module 100 for inflating or deflating all the airbags 120, the airbag control module 100 needs to employ a great many of airflow control components therein for controlling the inflating or deflating states of the airbags 120, including the plurality of electromagnetic intake valves 131 and the plurality of electromagnetic air release valves 132, this further causes an air pipe arrangement within the smart pillow device 10 is somewhat complex. Moreover, under a condition that several airbags 120 needs to be inflated or deflated by the total air pump 112 simultaneously, the airflow control mechanism of the airbag control module 100 needs to be very complicated, and the smart pillow device 10 is apt to suffering malfunction. Furthermore, the total air pump 112 is typically has an unduly big size and may have a loud operation noise, which is inappropriate for being integrated within the pillow body in accompany with the airbags 120 and the airbag control module 100, therefore, the total air pump 112 needs to be arranged in an accessional main control box 110, which further lower an integrity of the smart pillow device 10.

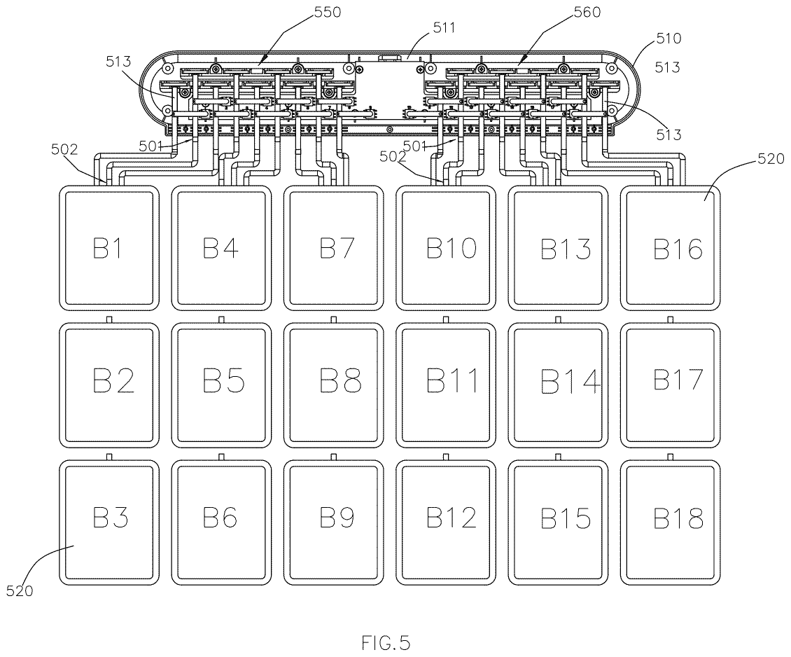

In view of the technical problems of the smart pillow device 10 as mentioned above, a new smart pillow device with antisnoring capability is further provided in the present application. FIG. 5 is a schematic diagram of a smart pillow device 50 according to an exemplary embodiment of the present application. The smart pillow device 50 includes a plurality of airbags 520 and an airbag control module 510 connected to the plurality of airbags 520. The airbags 520 and the airbag control module 510 may be arranged within a pillow body, which can be rested on for a user's head. Each of the airbags 520 includes an independent air inlet 502, the airbag control module 510 may have a box-like structure with a plurality of air outlets 501 arranged at a same side, each of which is connected to a respective air inlet 502 of the correspond airbags 520. The airbag control module 510 is configured for inflating or deflating the airbags 520 according to air pressures of the airbags 520 as well as a sleep posture of a user, so as to alleviate snoring phenomenon of the user during his or her sleep.

In the illustrated embodiment as shown in FIG. 5, the plurality of airbags 520 may be eighteen airbags 520 which are labeled with B1-B18 respectively, the airbags B1-B18 are arranged as an airbag array with three row and six columns. In particular, as an exemplary embodiment, the eighteen airbags B1-B18 may be grouped into a first airbag group B1-B9 and a second airbag group B10-B18, each of which is formed by nine airbags in a 3*3 matrix; it should be noted, however, in other embodiments, the number and arrangement of the airbags can have other design according to an actual need.

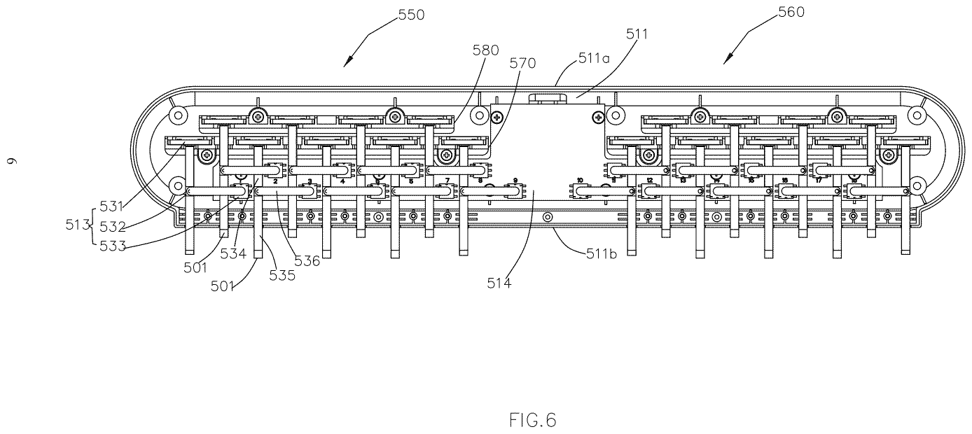

Referring also to FIG. 6, an enlarged view of the airbag control module 510 is shown. The airbag control module 510 includes a case 511 for providing an accommodating space, and a plurality of airflow control units 513 received in the accommodating space. The case 511 includes a first sidewall 511a and a second sidewall 511b opposite to each other, and the air outlets 501 may be formed at the second sidewall 511b. Moreover, each of the airflow control units 513 is independently connected to a respective one of the airbags 520 via a corresponding air outlet 501; in other words, eighteen independent airflow control unit 513 is provided in the case 511 in the present embodiment.

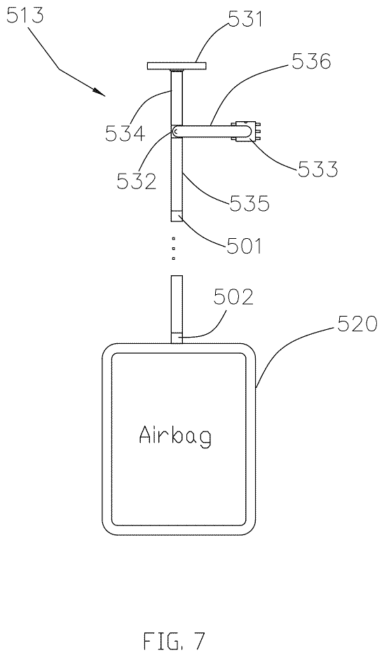

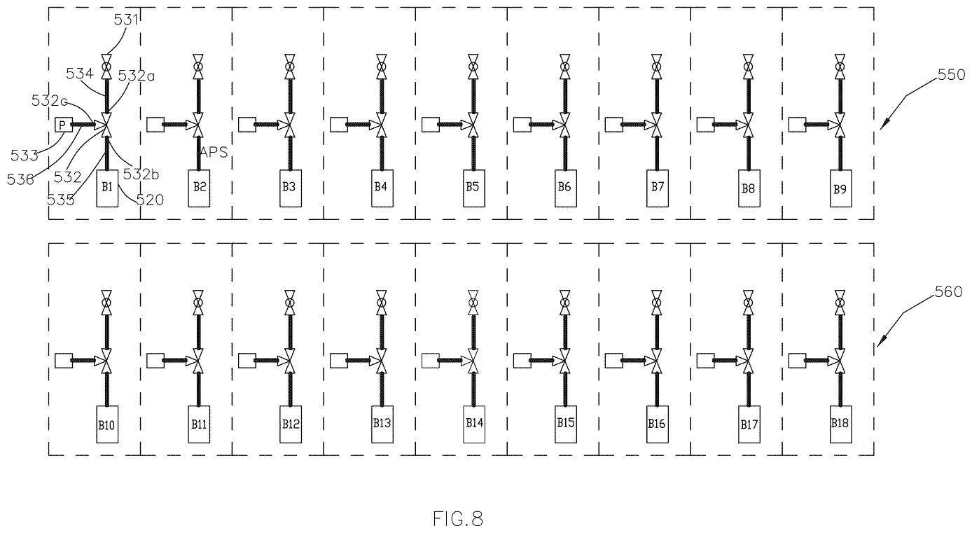

Referring also to FIG. 7 and FIG. 8, each of the airflow control units 513 includes a piezoelectric pump 531, a three-way air connector 532, and an air pressure sensor 533.

The piezoelectric pump 531 may for example be a micro piezoelectric ceramic air pump, which typically uses a converse piezoelectric effect to transform an electric signal into mechanical deformation of piezoelectric ceramic, and further causes change of air volume in an enclosed space to generate an independent airflow with a high pressure and a high speed through a vent thereof. By using the piezoelectric pump 531, under a condition that a corresponding airbag 120 needs to be inflated, for example, when an air pressure of the airbag 120 is not sufficient, the airflow control unit 513 may control the piezoelectric pump 531 to be pumped on to output an airflow to the airbag 120, such that the airbag 120 can be inflated to a sufficient height; in contrast, under a condition that the airbag 120 needs to be deflated, the piezoelectric pump 531 can be pumped off, so that the air in the airbag 120 can be deflated to make the airbag 120 to decrease to a desired height.

The three-way air connector 532 may be known as a tee-junction or a three-way pipe, which is configured for air communication among the piezoelectric pump 531, the airbag 520 and the air pressure sensor 533; the three-way air connector 532 may include a connector body having a first end 532a and a second end 532b opposite to each other, and a third end 532c formed at a top middle portion of the connector body. As illustrated in FIG. 8, the first end 532a of the three-way air connector 532 is connected to the vent of the piezoelectric pump 531 via a first connecting pipe 534 to receive the airflow generated by the piezoelectric pump 531. The second end 532b of the three-way air connector 532 is connected to the corresponding one of the airbags 520 via a second connecting pipe 535, to provide a deflate path of the airbag 520. The third end 532c of the three-way air connector 532 is connected to the air pressure sensor 533, to enable the air pressure sensor 533 to detect an air pressure of the airbag 520.

It should be noted that the three-way air connector (or tee-junction) 532 is meremly an example for providing the air connection among the piezoelectric pump 531, the airbag 520 and the air pressure sensor 533, in other embodiments, other forms of air connectors may be adapted as required.

The air pressure sensor 533 can be electrically mounted or fixed on a printed circuit board (PCB) 514 which is also arranged in the case 511, and in one embodiment, the air pressure sensors 533 of the plurality of airflow control units 513 can be mounted on a same PCB 514. The PCB 514 may, in addition, further include other control components such as a main controller or drive circuit to control or drive the piezoelectric pump 531 to operate according to an air pressure detection result or the air pressure sensor 533.

Moreover, to facilitate the connection between the three-way air connector 532 and the air pressure sensor 533, an arc-shaped pipe 536 can be connected between the third end 532c of the three-way air connector 532 and the air pressure sensor 533.

As mentioned above, in the present embodiment as illustrated in FIG. 5, in accordance with the grouping of the plurality of airbags 520 into the first airbag group B1-B9 and the second airbag group B10-B12, the plurality of airflow control units 513 can adaptively divided into a first airflow control set 550 and a second airflow control set 560. The first airflow control set 550 includes nine of the airflow control units 513 correspondingly connected to the first airbag group B1-B9, and is arranged at a left region within the case 511; the second airflow control set 560 includes the other nine of airflow control units 513 correspondingly connected to the second airbag group B10-B18, and is arranged at a right region within the case 511.

In each of the airflow control sets 550 and 560, the piezoelectric pumps 531 of the nine airflow control units 513 are arranged in two rows, which are staggered with each other; each of the piezoelectric pump 531 is disposed in an upright manner to be perpendicular to a bottom of the case 511, such that the vent of the piezoelectric pump 531 faces and is aligned with the air outlet 501 to make the first connecting pipe 534 and the second connecting pipe 535 be parallel to the bottom of the case 511. Moreover, the piezoelectric pumps 531 may be electrically connected to the PCB 514 to receive driving signals from the main controller or the driving circuit on the PCB 514, to perform pumping on or pumping off operation to inflate or deflate the corresponding airbags 520. With this configuration, the piezoelectric pumps 531 needs not to occupy a large space in the case 511, this can increase a space utilization to make the airbag control module 510 have a small size.

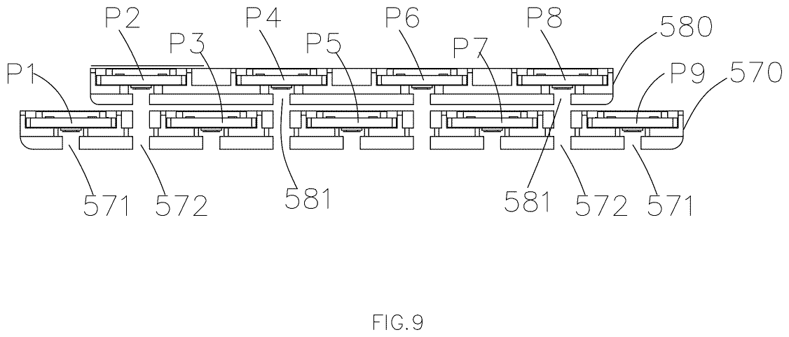

Taking the first airflow control set 550 as an example, referring also to FIG. 9, for ease to description, the piezoelectric pump 531 of the nine airflow control units 513 are labeled with P1-P9, namely, a first piezoelectric pump P1, a second piezoelectric pump P2, . . . , and a ninth piezoelectric pump P9.

The “odd-number” piezoelectric pumps P1, P3, P5, P7, P9 i are arranged in a first row, which is more adjacent to the second sidewall 511b with the air outlets 501 in relative to the “even-number” piezoelectric pumps P2, P4, P6, P8, while the “even-number” piezoelectric pumps P2, P4, P6, P8 are arranged in a second row which is more adjacent to the first sidewall 511a. For installation of the piezoelectric pumps P1-P9, a first bracket strip 570 and a second bracket strip 580, which are parallel to each other, are provided in the case 511. The first bracket strip 570 includes a plurality of bracket units for receiving the “odd-number” piezoelectric pumps P1, P3, P5, P7, P9, and each of the bracket units includes a first notch 571 aligned with the vent of the piezoelectric pump P1, P3, P5, P7, P9 received therein. The first notch 571 is configured for enable the first connecting pipe 534 to pass through the first bracket strip 570 and connected to the vent of the piezoelectric pump P1, P3, P5, P7, P9. Similarly, the second bracket strip 580 includes a plurality of bracket units for receiving the “even-number” piezoelectric pumps P2, P4, P6, P8, and each of the bracket units also includes a second notch 581 aligned with the vent of the piezoelectric pump P2, P4, P6, P8 received therein. In addition, the first bracket strip 570 may further include a plurality of third notches 572 arranged between any two adjacent bracket units thereof, each of the third notches 572 is aligned with a second notch 581 to enable the first connecting pipe 534 connected to the corresponding one of the “even-number” piezoelectric pumps P2, P4, P6, P8 to pass through the first bracket strip 570 and be connected to the three-way air connector 532.

In the present embodiment, each of the first notches 571, the second notches 581 and the third notches 572 may be a U-shaped notch, while in other embodiments, these notches 571, 581 and 572 can alternatively be formed as openings, holes or other shapes. Moreover, a distance between two bracket units for the piezoelectric pumps P1, P3, P5, P7, P9 in the first bracket trip 570 is preferred to be narrower, so long as to have sufficient width to form the third notches 572 therebetween. As such, the airbag control module 510 can further have a smaller size.

In the first airflow control set 550, referring back to FIG. 6, to make the component arrangement more compact to further improve an integrity of the airbag control module 510, the air pressure sensor 533 of the airflow control unit 513 may be disposed next to the three-way air connector 532 of an adjacent airflow control unit 513. In addition, the arc-shaped pipe 536 may be arranged to be perpendicular to the bottom of the case 511 and cross over the three-way air connector 532 of the adjacent airflow control unit 513, as illustrated in FIG. 6.

Moreover, as described above, since the piezoelectric pumps P1-P9 of the airflow control units 513 are arranged in two rows and staggered with each other, any two three-way air connectors 532 connected to two adjacent piezoelectric pumps P1-P9 are also located in a staggered manner. Accordingly, the arc-shaped pipes 536 in the airflow control units 513, that are respectively connected between the corresponding three-way air connectors 532 and the air pressure sensors 533, are also distributed alternately in two rows and parallel to each other.

Furthermore, at an air outlet region of the case 511, several batten bars may be provided to clip or fix the second connecting pipes 535 to the sidewalls 511b of the case 511, which can ensure the second connecting pipes 535 to be stably connected between the three-way air connectors 532 and the airbags 520 respectively. With this configuration, the second connecting pipes 535 can be protected from suffering shedding or undesired detachment during the user using the smart pillow device 50 for sleep. In one embodiment, three batten bars are provided for the nine airflow control units 513 in the first airflow control set 550, each of which is adapted for fixing three of the second connecting pipes 535 connected to the airbags 520 in a column.

The second airflow control set 560 has a same configuration as that of the first airflow control set 550, and thus details of the second airflow control set 560 can be referenced to the above description in accompany with FIGS. 5-9.

The smart pillow device 50 with antisnoring capability as provided in the above embodiments may have the following advantages. First of all, in the smart pillow device 50, each of the airbags 520 is inflated or deflated by using an independent airflow control unit 513 with a piezoelectric pump 531, thus the inflating or deflating operations of different airbags 520 can be implemented by the piezoelectric pumps 531 independently from each other and can be implemented simultaneously. This can reduce the complexity of the air pipe arrangement and simplify the airflow control mechanism, and further improve airbag inflating efficiency. Secondly, instead of a total air pump as described in the previous smart pillow device 10 in FIG. 2, each of the airflow control unit 513 in the smart pillow device 50 uses a respective piezoelectric pump 531, which has a low noise, low power consumption and occupy a small space, this makes it possible to be arranged into the pillow body in accompany with the airbags 520. Thirdly, the piezoelectric pumps 531 are received in an upright manner within the bracket strips 570 and 580 and to be arranged in two rows staggered with each other, and the arc-shaped pipes 536 in the case 511 are also staggered with each other, this can increase a space utilization to make the airbag control module 510 more compact, which is more adaptive to be received in the pillow body.

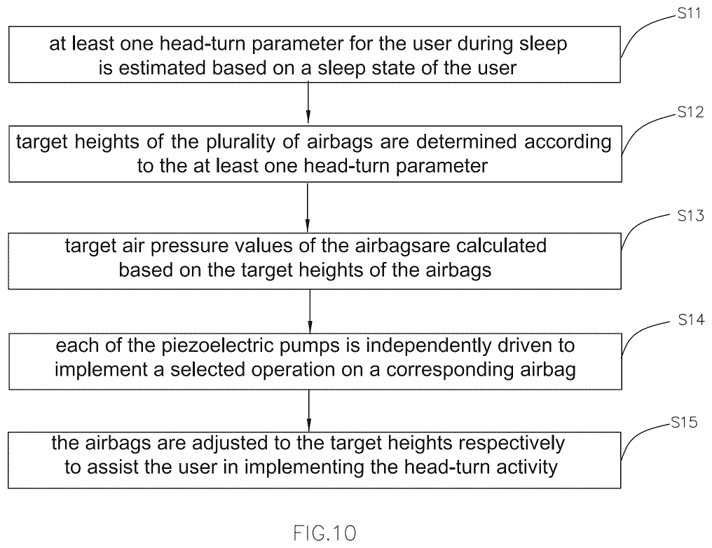

Based on the smart pillow device 50 as described in the above embodiments, the present application further provides a method for performing antisnoring for a user by using the smart pillow device 50. Referring to FIG. 10, a flow chart illustrating an operation process of the smart pillow device 50 is shown, which includes the following steps:

Step S11, at least one head-turn parameter for the user during sleep is estimated based on a sleep state of the user.

The at least one head-turn parameter may include an orientation parameter and/or an angle parameter of a head-turn activity to be performed by a user in order to alleviate physical snoring of the user. In an exemplary embodiment, the head-turn parameter may be estimated according to a sleep state of the user, which may for example be detected by other sensors arranged within the smart pillow device 50 or by software estimation according to a previous head-turn activity of the user.

Step S12, target heights of the plurality of airbags 520 are determined according to the at least one head-turn parameter.

For example, the orientation parameter and the angle parameter can be inputted to a head-tum model; the head-turn model may further use the orientation parameter and the angle parameter to determine a target height of each of the airbags 52. In this step, the target heights may be defined as heights of the airbags 520 in which the pillow body can be adjusted to a desired shape or profile, to enable a user to turn his or her head in a target orientation with a target angle during sleep.

Step S13, target air pressure values of the airbags 520 are calculated based on the target heights of the airbags 520.

A height of the airbag 520 typically has a correspondence relation with an air pressure thereof, in other words, if the air pressure of the airbag 520 is great, the height of the airbag 520 is also great, and vice versa. Therefore, based on the target heights as determined in the previous step S12, the target air pressure values of the plurality of airbags 520 can be calculated correspondingly in step S13.

Step S14, each of the piezoelectric pumps 531 is independently driven to implement a selected operation on a corresponding airbag 520, the selected operation includes one of an inflating operation, a deflating operation, and a pressure maintaining operation.

Upon calculation of the target air pressure values of the plurality of airbags 520, the current air pressure of the airbags 520 can also be received from the corresponding air pressure sensors 533, and therefore, it can further be determined that which of the airbags 520 needs to be inflated or deflated according to the current air pressure detected by the corresponding air pressure sensors 533. For example, if the calculated air pressure value of the airbag 520 is greater than the current air pressure detected by the air pressure sensor 533, it can be determined that the airbag 520 need to be inflated; if the calculated air pressure value of the airbag 520 is less than the current air pressure, it can be determined that the airbag 520 need to be deflated. In addition, if the calculated air pressure value of the airbag 520 is substantially equal to the current air pressure, it can be determined that the air pressure of the airbag 520 need to be maintained.

Therefore, each of the piezoelectric pumps 531 can be independently driven to enter a pump-on state to inflate the corresponding one of the airbags 520, or to enter a pump-off state to deflate the corresponding airbag 520, until the corresponding air pressure sensor 533 detects that the airbag 520 reaches the target air pressure value, and then the piezoelectric pumps 531 can be controlled to stop the inflating operation or the deflating operation on the airbag 520. In another aspect, if it is determined that a pressure maintaining operation needs to be performed on the airbag 520, the piezoelectric pump 531 can be driven to enter a low-frequency inflating state, to ensure that the air pressure of the airbag 520 is substantially stable in a predetermined range.

Step S15, the plurality of airbags 520 are adjusted to the target heights respectively to assist the user in implementing the head-turn activity for alleviating snoring during sleep.

When the airbags 520 are adjusted to the target heights respectively, the pillow body can have a desired shape or profile, which can enable the user to turn his or her head in the target orientation with the target angle during sleep; in this operation, the user may not be aware of his or her head-turn activity. As such, the physical snoring phenomenon of the user can be relieved, and therefore, the sleep quality of the user can be improved, so that the mental state and health of the user can also be improved.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A smart pillow device with antisnoring capability, comprising:
a plurality of airbags; and
an airbag control module comprising a case and a plurality of airflow control units received in the case, the plurality of airflow control units being connected to the plurality of airbags respectively;
wherein each of the airflow control unis comprises a piezoelectric pump, an air connector and an air pressure sensor, the piezoelectric pump is connected to a corresponding one of the airbags via the air connector, and the air pressure sensor is connected to the air connector for detecting an air pressure of the airbag; wherein the piezoelectric pump is driven to inflate or deflate the corresponding airbag independently according to an air pressure of the airbag.

2. The smart pillow device of claim 1, wherein the air connector is a three-way pipe comprising a first end connected to the piezoelectric pump via a first connecting pipe, a second end connected to the airbag via a second connecting pipe, and a third end connected to the air pressure sensor via an arc-shaped pipe.

3. The smart pillow device of claim 2, wherein the piezoelectric pumps of the plurality of airflow control units are arranged in two rows, and the two rows of the piezoelectric pumps are staggered with each other.

4. The smart pillow device of claim 3, wherein the airbag control module further comprises a first bracket strip and a second bracket strip provided in the case and parallel to each other, each of the first bracket strip and the second bracket strip comprises a plurality of bracket units for receiving a first row of the piezoelectric pumps and a second row of the piezoelectric pumps respectively.

5. The smart pillow device of claim 4, wherein each of the piezoelectric pumps are arranged in a respective bracket unit in an upright manner and is perpendicular to a bottom of the case, with a vent thereof facing the first end of the corresponding three-way pipe.

6. The smart pillow device of claim 5, wherein the first bracket strip and the second bracket strip comprise a plurality of first notches and a plurality of second notches respectively, each of the first notches and the second notches is aligned with the vent of the corresponding piezoelectric pump for connecting the first connecting pipe to the vent of the piezoelectric pump.

7. The smart pillow device of claim 6, wherein the first bracket strip further comprises a plurality of third notches, each of the third notches is arranged between two adjacent bracket units in the first bracket strip and is aligned with a corresponding one of the second notches in the second bracket strip, the third notches are configured for enabling the first connecting pipes connected to the piezoelectric pumps in the second bracket strip to pass through the first bracket strip.

8. The smart pillow device of claim 2, wherein the first end and the second end of the three-way pipe are formed at two opposite ends of a connector body, to make the first connecting pipe and the second connecting pipe connected thereto are parallel to the bottom of the case, and the third end of the three-way air connector is formed at a top middle portion of the connector body to make the arc-shaped pipe connected between the third end of the three-way pipe and the air pressure sensor to be perpendicular to the bottom of the case.

9. The smart pillow device of claim 8, wherein the air pressure sensor of the airflow control unit is disposed next to a three-way pipe of an adjacent airflow control unit, and the arc-shaped pipe of the airflow control unit crosses over the three-way pipe of the adjacent airflow control unit to be connected with the air pressure sensor.

10. The smart pillow device of claim 9, wherein any two three-way pipe connected to two adjacent piezoelectric pumps are arranged in a staggered manner, to make the arc-shaped pipes connected to the plurality of three-way air connectors are distributed alternately in two rows and parallel to each other.

11. The smart pillow device of claim 3, wherein the airbag control module further comprises a plurality of batten bars, each of the batten bars is configured for clipping or fixing several second connecting pipes to the case at an air outlet region.

12. The smart pillow device of claim 1, wherein the airflow control module is received in a pillow body in accompany with the plurality of airbags, the plurality of airbags comprises a first airbag group and a second airbag group arranged as an airbag array, the plurality of airflow control units comprise a first airflow control set and a second airflow control set disposed at different regions in the case and for inflating or deflating the first airbag group and the second airbag group respectively, wherein air outlets of the first airflow control set and the second airflow control set are extended from a same sidewall of the case.

13. The smart pillow device of claim 1. wherein the piezoelectric pump is a micro piezoelectric ceramic air pump using converse piezoelectric effect to transform an electric signal into mechanical deformation of piezoelectric ceramic to generate an airflow to the corresponding airbag.

14. The smart pillow device of claim 13, wherein the piezoelectric pumps and the air pressure sensors of the plurality of airflow control units are electrically mounted to a printed circuit board, and the printed circuit board further comprises a main controller or a drive circuit for driving the piezoelectric pumps to pump on or pump off according to the air pressures detected by the air pressure sensors so as to inflate or deflate the airbags to assist a user in implementing a head-turn activity during sleep.

15. A method for performing antisnoring for a user by using a smart pillow device, the smart pillow device comprising a plurality of airbags and a plurality of airflow control units, each of the airflow control units comprising a piezoelectric pump and an air pressure sensor connected to a corresponding airbag, the method comprising:
estimating at least one head-turn parameter for the user during sleep:
determining target heights of the plurality of airbags based on the at least one head-turn parameter, and calculating target air pressure values in accordance with the target heights of the airbags; and
driving each of the piezoelectric pumps independently to inflate or deflate a corresponding one of the airbags according to the target air pressure value.

16. The method of claim 15, wherein at least one head-turn parameter comprises an orientation parameter and/or an angle parameter of a head-turn activity to be performed by a user.

17. The method of claim 15, wherein the driving each of the piezoelectric pumps independently to inflate or deflate a corresponding one of the airbags comprises:
receiving a current air pressure of the airbag detected by the air pressure sensor,
determining whether the airbag need to be inflated or deflated based on the target air pressure value and the current air pressure of the airbag:
performing a corresponding one of an inflating operation, a deflating operation, or a pressure maintaining operation on the airbag by the piezoelectric pump.

18. The method of claim 17, wherein
upon determining that the target air pressure value of the airbag is greater than the current air pressure, the piezoelectric pump is driven to pump on to inflate the airbag until the air pressure sensor detecting that the air pressure of the airbag reaches the target air pressure value; or
upon determining that the target air pressure value of the airbag is less than the current air pressure, the piezoelectric pump is driven to pump off to deflate the airbag until the air pressure sensor detecting that the air pressure of the airbag reaches the target air pressure value; or
upon determining that the target air pressure value of the airbag is substantially equal to the current air pressure, the piezoelectric pump is driven to enter a low-frequency inflating state to make that the air pressure of the airbag be substantially stable in a predetermined range.

19. The method of claim 18, wherein after being performed with the inflating operation, the deflating operation, or the pressure maintaining operation by the piezoelectric pumps, the airbags are adjusted to the target heights respectively to make a pillow body have a desired profile to assist the user in implementing the head-turn activity for alleviating snoring during sleep.

20. A smart pillow device with antisnoring capability, comprising:
a plurality of airbags; and
an airbag control module comprising a case and a plurality of airflow control units received in the case, the plurality of airflow control units being connected to the plurality of airbags respectively;
wherein each of the airflow control units comprises an air connector and an air pressure sensor, the air connector is connected to a corresponding one of the airbags and the air pressure sensor; wherein the air pressure sensor is configured for detecting an air pressure of the airbag, the air connector is configured for transmitting an airflow provided by an air source to the airbag to inflate the airbag independently when it is determinized that an air pressure of the airbag is less than a target air pressure.
